(19) United States
(12) Patent Application Publication
(10) Pub. No.: US 2016/0053255 A1
(43) Pub. Date: Feb. 25, 2016

(54) METHOD FOR TRANSFECTING A EUKARYOTIC CELL

(71) Applicant: QIAGEN GMBH, Hilden (DE)

(72) Inventors: Peter Hahn, Hilden (DE); Anja Grewe, Hilden (DE)

(21) Appl. No.: 14/918,228

(22) Filed: Oct. 20, 2015

Related U.S. Application Data
(63) Continuation of application No. 13/697,188, filed on Dec. 17, 2012, now abandoned.

(30) Foreign Application Priority Data
May 10, 2010 (EP) 10004912.1

(51) Int. Cl. C12N 15/11

(52) U.S. Cl. CPC C12N 15/11

(57) ABSTRACT

A method for transfecting a eukaryotic cell in which:
a composition that includes at least one saccharide and at least one protein and optionally a salt and/or a pH-stabilizing component is contacted with a nucleic acid and a transfection reagent;
a complex of nucleic acid and transfection reagent is formed by interaction with the composition;
the complex is dried;
the dried complex is stored for a period of time;
after storing, the dried complex is taken up into a liquid; and
the complex taken up into the liquid is transfected into the eukaryotic cell.

### CROSS-REFERENCE TO RELATED APPLICATIONS

This application is a continuation of co-pending U.S. application Ser. No. 13/697,188, filed Dec. 17, 2012, which is a U.S. national stage of PCT/EP2011/057304, filed May 6, 2011, and claims priority to EP 10004912.1, filed May 10, 2010.

### BACKGROUND OF INVENTION

1. Field of Invention

The present invention relates to a method for transfecting a eukaryotic cell, wherein a composition which comprises at least one saccharide and at least one protein and optionally a salt and/or a pH-stabilizing component is contacted with a nucleic acid and a transfection reagent, a complex of nucleic acid and transfection reagent is formed by interaction with the composition, and the complex is transfected into a eukaryotic cell.

2. Brief Description of Related Art

From the state of the art, many different methods are known for introducing foreign nucleic acids, in particular foreign DNA and foreign RNA, into a cell by transfection. In particular, in the state of the art cationic polymers which enter into complexes with the nucleic acid to be transfected are described.

These cationic polymers for example include polyethylenimine (PEI) and homopolymers of basic amino acids such as for example poly-L-lysine. Dendritic cationic polymers, such as for example polyamidoamines (PAMAMs), and polyamides, polyallylamines, cationic methacrylates, chitosan or poly(D,L-lactide coglycolide (PLGAs) have also been described as suitable cationic polymers for transfecting cells.

Furthermore, in the state of the art, complexes of lipid-based reagents and nucleic acids are described for introducing foreign nucleic acids into a cell by transfection. These for example include cationic lipid vesicles onto the surface whereof the nucleic acid is complexed. Examples of lipid-based reagents are DOTMA (N-[1-(2,3-dioleyloxy)propyl]-N,N,N-trimethylammonium chloride) and mixtures of DOTMA with phospholipids, from which positively charged liposomes are produced, onto which the negatively charged nucleic acid binds.

Commercially available transfection reagents such as HiPerFect (QIAGEN), Attractene (QIAGEN) and HT-Fect (QIAGEN) also often consist of lipid-based reagents, with which the nucleic acid to be transfected is complexed.

Usually the complexes of transfection reagent and nucleic acid to be transfected are only stable for a short time span, of about one to a few hours, so that these complexes can in general only be prepared directly before the transfection. Storage of these complexes over a longer period or drying (lyophilization) of these complexes is not possible without the efficiency of the subsequent transfection being markedly reduced, or a transfection being no longer possible at all.

### BRIEF SUMMARY OF THE INVENTION

The present invention is based on the problem of overcoming the disadvantages arising from the state of the art, and providing a method for transfecting a eukaryotic cell.

The present invention is also based on the problem of providing a method for transfecting a eukaryotic cell wherein the complex of nucleic acid and transfection reagent is stabilized such that it can be subjected to a storage step.

The present invention is also based on the problem of providing a composition which makes it possible to stabilize a complex of a nucleic acid and a transfection reagent, so that this complex is storable.

The problem is solved by the subject matter defined in the claims.

Thus the present invention provides a method for transfecting a eukaryotic cell, comprising the following process steps:
a. Contacting a composition comprising at least one saccharide and at least one protein, and optionally a salt and/or a pH-stabilizing component, with a nucleic acid and a transfection reagent;
b. Formation of a complex of nucleic acid and transfection reagent by interaction with the composition from step a;
b. Transfection of a eukaryotic cell by means of the complex from step b.

The present invention further relates to the use of a composition for stabilizing a complex of a nucleic acid and a transfection reagent, characterized in that the composition comprises at least one saccharide and at least one protein, and optionally a salt and/or a pH-stabilizing component.

The present invention further relates to a composition for storing a complex of a nucleic acid and a transfection reagent, characterized in that it comprises at least one saccharide and at least one protein and a nucleic acid and a transfection reagent, and optionally a salt and/or a pH-stabilizing component.

### BRIEF DESCRIPTION OF THE DRAWINGS

FIG. 1 shows the analysis of a gene-silencing effect by means of qPCR after transfecting HeLa S3 cells with an siRNA directed against lamin after 2 days' storage at 4° C. Before the storage, different compositions were added to the siRNA and the transfection reagent.

FIG. 2 shows the analysis of a gene-silencing effect by means of qPCR after transfecting HeLa S3 cells with an siRNA directed against lamin after 5 days' storage at 4° C. or room temperature. Before the storage, different compositions were added to the siRNA and the transfection reagent.

FIG. 3 shows the analysis of a gene-silencing effect by means of qPCR after transfecting HeLa S3 cells with an siRNA directed against lamin at different concentrations after 2 days' storage at 4° C. Before the storage, different compositions were added to the siRNA and the transfection reagent. For comparison, freshly prepared transfection complexes were transfected.

FIG. 4 shows the analysis of a gene-silencing effect by means of qPCR after transfecting HeLa S3 cells with an siRNA directed against lamin at different concentrations after 5 days' storage at 4° C. Before the storage, different compositions were added to the siRNA and the transfection reagent.

FIG. 5 shows the analysis of the survival rate of HeLa S3 cells which had been transfected with an siRNA which triggers cell death. Dried complexes of transfection reagent were transfected with siRNA in comparison to freshly prepared complexes. In no case was the composition according to the invention added to the complex.

FIG. 6 shows the analysis of a gene-silencing effect by means of qPCR after transfecting HeLa S3, MCF-7 or HepG2 cells with two different siRNAs (WASL_{—}6 (SEQ ID NO: 11) and WASL_{—}9 (SEQ ID NO: 12)), which are directed against WASL. The transfection complexes were dried together with the composition according to the invention, and stored for 2 months at 4° C., room temperature or 37° C.

FIG. 7 shows the analysis of a gene-silencing effect by means of qPCR after transfecting HeLa S3 and MCF-7 cells with an siRNA directed against lamin. The composition according to the invention was added to the transfection complex of siRNA and transfection reagent and subjected to 3 or 5 freeze-thaw cycles before the cells were transfected. As a control, fresh transfection complexes were transfected.

FIG. 8 shows the analysis of a gene-silencing effect by means of qPCR after transfecting HeLa S3 cells with an siRNA which is directed against CDC2. The transfection complex was formed with HT-Fect (QIAGEN) or HiPerFect (QIAGEN) as transfection reagent. To the transfection complex of siRNA and transfection reagent were added various compositions. One part of the complex with composition was transfected fresh, a further part dried and taken up again into water, and a third part was stored at −20° C.

FIG. 9 shows the quantitative determination of the GFP-positive cells in % of all assayed cells after transfecting HeLa S3 and MCF-7 cells with a plasmid which codes for a Green Fluorescent Protein (GFP). The complex of plasmid and transfection reagent was dried in vacuo together with the composition according to the invention and stored at 4° C.

### DETAILED DESCRIPTION OF THE INVENTION

Entirely surprisingly, it was found that by means of this composition not only can eukaryotic cells be transfected very efficiently with a nucleic acid with low cytotoxicity, but that in addition this composition stabilizes the complex of nucleic acid and transfection reagent such that the complex of nucleic acid and transfection reagent becomes storable.

For this reason, a storage step can be inserted following step b of the method according to the invention.

In the sense of the present invention, transfection is understood to mean the introduction of foreign nucleic acid into a eukaryotic cell by means of a transfection reagent.

The eukaryotic cell, into which a nucleic acid is introduced in the method according to the invention can be a cell culture cell, but this can also be a cell or a tissue structure which was obtained directly from a eukaryotic organism.

In a preferred embodiment the eukaryotic cell is a cell culture cell.

The eukaryotic cell can be a human cell, a cell from a mammal, from a vertebrate or an invertebrate. However, the eukaryotic cell can also be of plant origin or derive from a fungus. The eukaryotic cell can also be a single-cell organism, such as for example a yeast.

The nucleic acid which in process step a of the method according to the invention is contacted with a transfection reagent and a composition according to the invention can be a ribonucleic acid (RNA), desoxyribonucleic acid (DNA) or even a mixed form, a ribonucleic acid being preferred.

Particularly preferred is a short ribonucleic acid such as siRNA, miRNA, piRNA, snRNA or snoRNA, and the short ribonucleic acid is still more particularly preferably an siRNA.

The term siRNA is understood to mean a double-stranded ribonucleic acid which has a length of about 18 to 30 nucleotides and which can be incorporated into the so-called RISC enzyme complex (RNA-induced silencing complex).

In the general case, it can be any type of nucleic acid which is an N-glycoside or C-glycoside of a purine or pyrimidine base or of a modified purine or pyrimidine base. The nucleic acid can thus also contain bases which do not occur in nature, such as for example inosine or ribo-thymidine.

Apart from the base itself, the sugar residue can also be modified. Nucleic acids with modified linkage of the subunits, such as phosphorothioate or amidate linkages, are included.

The nucleic acid which in step a of the method according to the invention is contacted with a transfection reagent and a composition according to the invention can be single, double or multistrand, linear or circular. It can correspond to a molecule occurring in a cell, such as for example genomic DNA, messenger RNA (mRNA) or miRNA, or be created in vitro such as for example complementary DNA (cDNA), siRNA or synthetic nucleic acids.

The nucleic acid can consist of few nucleotides, preferably eight or more nucleotides, such as for example oligonucleotides, siRNA or miRNA, several hundred nucleotides up to several thousand nucleotides, for example comprising certain expression vectors.

In a preferred embodiment, the nucleic acid contains no coding information for a polypeptide, but rather is capable of binding complementarily to an mRNA, such as occurs in the cell to be transfected, or to an mRNA introduced from outside into this cell directly or indirectly via an expression vector and reducing the translation of this mRNA into a polypeptide.

Transfection is generally understood to mean the process of introducing a nucleic acid into a eukaryotic cell. As well as methods such as electroporation, calcium phosphate precipitation and introduction of the nucleic acid by means of a particle gun, methods wherein the nucleic acid is introduced into the cell by means of a transfection reagent are most usual today.

Suitable transfection reagents in the sense of the present invention are for example highly branched organic molecules, so-called dendrimers, cationic polymers such as for example polyethylenimine (PEI), homopolymers of basic amino acids such as for example poly-L-lysine, polyamides, polyallylamines, cationic methacrylates, chitosan or poly(D,L-lactide coglycolide (PLGAs).

A preferred group of transfection reagents are lipid-based transfection reagents. These for example include cationic lipid vesicles on the surface whereof the nucleic acid is complexed. Examples of lipid-based reagents are DOTMA (N-[1-(2,3-dioleyloxy)propyl]-N,N,N-trimethylammonium chloride) and mixtures of DOTMA with phospholipids, from which positively charged liposomes are produced, onto which the negatively charged nucleic acid binds.

Commercially available transfection reagents such as HiPerFect (QIAGEN), Attractene (QIAGEN) and HT-Fect (QIAGEN) also often consist of lipid-based reagents, with which the nucleic acid to be transfected is complexed and are likewise preferred transfection reagents in the sense of the present invention. Many other suitable transfection reagents from the various chemical classes are known to those skilled in the art.

The composition with which the nucleic acid and the transfection reagent are contacted in step a of the method according to the invention comprises at least one saccharide and at least one protein.

Here the saccharide can be a simple sugar (monosaccharide), a double sugar (disaccharide) a triple sugar (trisaccharide), a multiple sugar (polysaccharide) or mixtures of at least two different saccharides.

In principle, according to the invention all saccharides or mixtures of saccharides are suitable for the method according to the invention, as long as the saccharides as such do not have a toxic action on 100% of the eukaryotic cells used in the method.

By simple preliminary experiments by optical monitoring of the eukaryotic cells, those skilled in the art can establish which saccharides have a toxic effect on the eukaryotic cell used by them and to what extent, since the morphology of the eukaryotic cell changes as a result of cytotoxic effects.

Examples of suitable monosaccharides are glucose, mannose, fructose, ribose, desoxyribose, galactose, fucose or rhamnose. Those skilled in the art are familiar with other suitable monosaccharides.

Examples of suitable disaccharides are saccharose, lactose, lactulose, maltose or trehalose. Those skilled in the art are familiar with other suitable disaccharides.

Examples of suitable trisaccharides are melezitose, raffinose or umbelliferose. Those skilled in the art are familiar with other suitable trisaccharides.

As a polysaccharide, starch can for example be used. Those skilled in the art are familiar with other suitable polysaccharides.

In a preferred embodiment, the saccharide or mixture of saccharides are disaccharides.

In a particularly preferred embodiment, the disaccharide is saccharose or trehalose or a mixture of the two.

In a quite especially preferred embodiment, the disaccharide is saccharose.

The concentration of the saccharide can lie in any range up to the solubility limit thereof in the buffer used, provided that all cells must not be killed by cytotoxic effects due to the concentration used.

By simple preliminary experiments by optical monitoring of the morphology of the eukaryotic cell, those skilled in the art can establish whether the concentration used of the saccharide used by them causes a change in the morphology of the cell.

In a preferred embodiment, the concentration of the saccharide or of the mixture of saccharides is selected such that more than half of all eukaryotic cells exhibit no change in their morphology caused by cytotoxicity.

In a further preferred embodiment, the saccharide or the mixture of saccharides are present at a concentration of 0.01M to 2M, at which the change in the morphology caused by the cytotoxicity affects no more than half of all eukaryotic cells.

In a still more preferred embodiment, the saccharose or the trehalose or the mixture of the two are present at a concentration of 0.01M to 2M, still more preferably at a concentration of 0.05M to 1M, still more preferably at a concentration of 0.1 to 0.5M, and still more preferably at a concentration of 0.1M.

In a still further quite especially preferred embodiment, the saccharose is present at a concentration of 0.01M to 2M, still more preferably at a concentration of 0.05M to 1M, still more preferably at a concentration of 0.1 to 0.5M, and still further quite especially preferably at a concentration of 0.1M.

The composition with which the nucleic acid and the transfection reagent are contacted in step a of the method according to the invention comprises at least one saccharide and at least one protein.

Here the protein can in principle be a protein consisting of one or more subunits or a polypeptide consisting of several amino acids. However, it can also be a mixture of two or more proteins and/or polypeptides. Here, so that nucleic acid and transfection reagent can form a complex, the protein or the mixture must be selected such that it does not because of its charge prevent the interaction between the nucleic acid and the transfection reagent.

Furthermore, the protein or the mixture must be selected such that it triggers no effect in the eukaryotic cell which as a reaction to this protein prompts the cell to up- or down-regulate certain cellular processes. Rather, the protein or the mixture must be selected such that the cellular processes of the cell largely remain unaffected.

Hence proteins such as for example BSA or other albumins or mixtures such as fetal calf serum are suitable for the method according to the invention.

In a preferred embodiment, the protein is BSA.

The concentration of the protein used should lie in a range wherein it is soluble in the composition according to the invention and does not precipitate and triggers no cytotoxic effects.

In a preferred embodiment, the content by weight of the protein in the composition according to the invention is 0.1% to 5%, in a quite especially preferred embodiment its content by weight is 0.5% to 2%, and still more especially preferably its content by weight is 1%.

In a particularly preferred embodiment, the protein in the composition according to the invention is BSA with a content by weight of 0.1% to 5%, still more preferably with a content by weight of 0.5% to 2%, still more especially preferably with a content by weight of 1%.

A salt can optionally be added to the composition according to the invention. The salt can serve to promote or to stabilize the formation of the complex between nucleic acid and transfection reagent or also to promote or to stabilize the interaction between the composition according to the invention and the complex of nucleic acid and transfection reagent, for example, in that the complex is stabilized by charge equalization.

A further function of the salt can also be to stabilize the reaction medium in general or to contribute to a higher efficiency of the transfection.

Examples of suitable salts are halides, phosphates, nitrates or sulfates of alkali or alkaline earth metals or ammonium ions. However, the salts of organic acids such as acetates or citrates are also suitable.

Those skilled in the art are familiar with many other suitable salts which are capable of promoting or stabilizing the formation of the complex as described above or of stabilizing the reaction medium in general or contributing to a higher efficiency of the transfection.

In the composition used in the method according to the invention, a pH-stabilizing component can optionally be added. Through stabilizing the pH, this component can serve to promote or to stabilize the formation of the complex between nucleic acid and transfection reagent, or also to promote or to stabilize the interaction between the composition according to the invention and the complex of nucleic acid and transfection reagent.

A further function of the pH-stabilizing component can also be to bring the pH for the subsequent transfection into a suitable range.

Examples of suitable pH-stabilizing components are HBS, PBS and Optimem (Life Technologies).

After the composition containing at least one saccharide and at least one protein and optionally a salt and/or a pH-stabilizing component has been contacted in step a of the method according to the invention with a nucleic acid and a transfection reagent, in step b of the method according to the invention a complex of nucleic acid and transfection reagent is formed. This complex is based on non-covalent interactions, mainly on ionic interactions, between the nucleic acid and the transfection reagent. This complex of transfection reagent and nucleic acid interacts non-covalently with the composition from step a of the method according to the invention, during which this interaction has a stabilizing effect.

In step c of the method according to the invention, a eukaryotic cell is transfected by means of the complex from step b of the method according to the invention.

Entirely surprisingly, it was found that the composition according to the invention makes it possible to make the complex of nucleic acid and transfection reagent storable, and that after storage it is possible to transfect a eukaryotic cell with this complex.

For this reason it is possible, after the formation of a complex of nucleic acid and transfection reagent by interaction with the composition from step a of the method according to the invention, to store the complex after step b, before a eukaryotic cell is transfected in step c by means of the complex from step b.

Storing the complex can be effected here such that the complex is dried for storage. For this, the liquid is removed from the complex of nucleic acid and transfection reagent which interacts with the composition according to the invention.

The removal of the liquid can occur by drying of the complex with the composition in vacuo. This can for example be effected by means of a vacuum bell jar or else by vacuum centrifugation or by means of a freeze-drying unit.

The drying can however also take place at normal atmospheric pressure. For this, the complex with the composition according to the invention can be allowed to stand at room temperature in an open vessel until the liquid has evaporated. It is also possible to accelerate the evaporation process by heating the vessel with the complex and the composition according to the invention at temperatures above room temperature. Here, however, the temperature must remain in a range wherein neither the nucleic acid nor the transfection reagent are thermally destroyed or irreversibly denatured. Thereby it is possible, within a very short time and with very low technical expenditure, to generate complexes of transfection reagent and nucleic acid which can be stored over a prolonged period and which after the storage can be successfully transfected into a eukaryotic cell.

If the complex is dried for storage, it is necessary that before transfecting a eukaryotic cell the complex with the composition according to the invention be again taken up into a liquid.

This liquid can be water, but here it can also be a buffer which contains salts and/or a pH-stabilizing component. Finally, this liquid can also be a component which increases the transfection efficiency. Here it can for example be an organic molecule which is adapted to the particular transfection reagent and increases the transfection efficiency in cooperation with the transfection reagent. This molecule is also described as an enhancer.

Irrespective of whether the complex with the composition according to the invention is dried for storage or remains in a liquid state, the storage can take place at room temperature (20° C. to 25° C.). This has the major advantage that for the method according to the invention it is not necessary to have to store the complex with the composition according to the invention in a refrigerator, cold room or freezer chest, so that valuable space can be utilized for more sensitive substances. Also it is thus easily possible to ship the complex with the composition according to the invention at room temperature. This saves costs since shipping in a cool box can be dispensed with. Moreover, it is thereby ensured that a delay in shipping, during which the samples thaw out, the complex of transfection reagent and nucleic acid is still very well suited for transfecting a eukaryotic cell.

It is however also possible to effect the storage at a temperature which lies below room temperature.

Thus for example it is possible to effect the storage at temperatures of 2° C. to 8° C. in the refrigerator or a cold room.

Likewise it is for example possible to effect the storage at temperatures below 0° C., for example by cooling to −20° C. or temperatures which lie further below this temperature.

If the temperatures for the storage lie below the freezing point of the liquid in which the complex and the composition according to the invention are present, then the liquid must again be present as liquid before transfecting a eukaryotic cell.

For the storage at temperatures below room temperature it is also not important whether the complex with the composition according to the invention is dried or remains in the liquid state.

Storing the complex with the composition according to the invention can here take place for a short period, for example for several hours or a few days, but it is also possible to store the complex for several weeks or months.

Here, the lower is the selected storage temperature, the longer is the possible storage time.

In step c of the method according to the invention transfecting a eukaryotic cell is performed. Here it is possible to mix the complex with the composition according to the invention directly together with the cells to be transfected, provided that the complex was not dried and the storage took place above the freezing point of the complex.

If the storage took place below the freezing point, the transfection can be performed directly after thawing, since the complex of transfection reagent and nucleic acid was stabilized by the composition according to the invention such that a successful transfection is possible without a further process step.

If the storage took place in the dried state, the transfection can take place directly after uptake of the complex with the composition according to the invention in water or in another suitable liquid, since the complex of transfection reagent and nucleic acid was stabilized by the composition according to the invention such that a successful transfection is possible without a further process step.

It is however also possible that before the transfection still further components are added to the complex with the composition according to the invention, for example salts, pH-stabilizing components or enhancers of the transfection reagent.

Those skilled in the art are familiar with such suitable components and enhancers.

In a preferred embodiment, no further components are added to the complex with the composition according to the invention before transfecting a eukaryotic cell, since the composition according to the invention together with the complex of nucleic acid and transfection reagent contains all components necessary for an efficient transfection, and thus further process steps can be dispensed with.

If the complex with the composition according to the invention had been stored in the dried state, then in a preferred embodiment this is taken up in water.

### EXAMPLES

The following examples are intended to further illustrate the invention, without this being limited to the practical examples.

Sequences Used:

Primers:

3Lamin3815:
(SEQ ID NO: 1)
5′-GCATCTCATCCTGAAGTTGCTT
3Lamin8:
(SEQ ID NO: 2)
5′-CAAGAAGGAGGGTGACCTGA
CDC2F:
(SEQ ID NO: 3)
5′-CAGGATTTTCAGAGCTTTGGG
CDC2R:
(SEQ ID NO: 4)
5′-GCTGGATCATAGATTAACATTTTCGAG
WASLF:
(SEQ ID NO: 5)
5′-CGACAAAGGAAATCTGAGAAA
WASLR:
(SEQ ID NO: 6)
5′-GGGAGATGTTGTTGACTTGTG
GAPDHF:
(SEQ ID NO: 7)
5′-GCCTCAAGATCATCAGCAAT
GAPDHR:
(SEQ ID NO: 8)
5′-GAGTCCTTCCACGATACCAA

siRNAs (Guide Strand):

Lamin A/C:
(SEQ ID NO: 9)
5′-rGrCrGrCrCrArGrArArUrGrGrArGrArUrGrArUTT
Cdc2_641:
(SEQ ID NO: 10)
5′-rUrUrGrArGrUrArArCrGrArGrCrUrGrArCrCrCrCrA
WASL_6:
(SEQ ID NO: 11)
5′-rUrUrUrUrArArArCrUrCrCrCrUrUrGrUrUrGAT
WASL_9:
(SEQ ID NO: 12)
5′-rUrUrUrGrUrUrUrCrCrUrGrCrArGrUrArGrUrUGG
Hs_plk1:
(SEQ ID NO: 13)
5′-rUrArUrUrCrArUrUrCrUrUrCrUrUrGrArUrCrCrGrG
Hs_Ubb:
(SEQ ID NO: 14)
5′-rGrGrCrCrArArGrArUrCrCrArArGrArUrArArATT
NSC:
(SEQ ID NO: 15)
5′-rUrUrUrGrUrArArUrCrGrUrCrGrArUrArCrCrCrUrG

### Example 1

### Stabilization of a Complex of Transfection Reagent and siRNA with Various Compositions with Storage of the Complex for 2 Days at 4° C.

20 nM of lamin A/C (SEQ ID NO: 9) or 20 nM Non Silencing Control (NSC (SEQ ID NO: 15), Allstar neg.) siRNA and 3 μl of the transfection reagent HiPerfect (QIAGEN) and different stabilizers of the complex of transfection reagent and nucleic acid were added to a total volume of 100 μl of buffer HBS (20 mM Hepes, 150 mM NaCl, pH 7.4).

These were (final concentration in each case):

0.1M saccharose, 1% (w/v) BSA

0.1M saccharose, 1% (w/v) BSA, 0.05 mg/ml Gly-Gly

0.1M saccharose, 5% (w/v) BSA

0.1M saccharose, 5% (w/v) BSA, 0.05 mg/ml Gly-Gly

0.2M saccharose, 1% (w/v) BSA

0.2M saccharose, 1% (w/v) BSA, 0.05 mg/ml Gly-Gly

0.2M saccharose, 5% (w/v) BSA

0.2M saccharose, 5% (w/v) BSA, 0.05 mg/ml Gly-Gly

DMEM (Invitrogen)+serum

The complexes with the stabilizers were incubated for 5 to 10 minutes at room temperature and then stored for 48 hours at 4° C.

After storage, the preparations were added dropwise to 60,000 HeLa S3 cells in each case and transfected as per the HiPerFect-fastforward protocol (QIAGEN).

After incubation of the cells for 48 hours at 37° C. in 5% CO₂, the RNA was isolated by means of the RNeasy protocol (QIAGEN).

In the successfully transfected cell, the expression of the lamin mRNA is reduced by the transfected lamin A/C (SEQ ID NO: 9) siRNA. For the determination of the transfection efficiency, the reduction expression of the lamin mRNA in relation to the expression of the GAPDH mRNA, the expression whereof remains unaffected by the transfection, was determined by means of a quantitative RT-PCR. For this, 2 μl of a 1:20 dilution of the isolated RNA was reverse transcribed and then amplified as per the QuantiFast SYBR Green One-Step RT-PCR protocol (Qiagen) by means of the primers 3Lamin3815 (SEQ ID NO: 1) and 3Lamin8 (SEQ ID NO: 2) and GAPDHF (SEQ ID NO: 7) and GAPDHR (SEQ ID NO: 8) respectively in a 10 μl preparation. The reverse transcription and the amplification took place in an ABI 7900 Cycler (Applied Biosystems), and the reaction conditions were:

10 mins 50° C.

5 mins 95° C.
Then 40 cycles
   10 secs 95° C.
   30 secs 60° C.

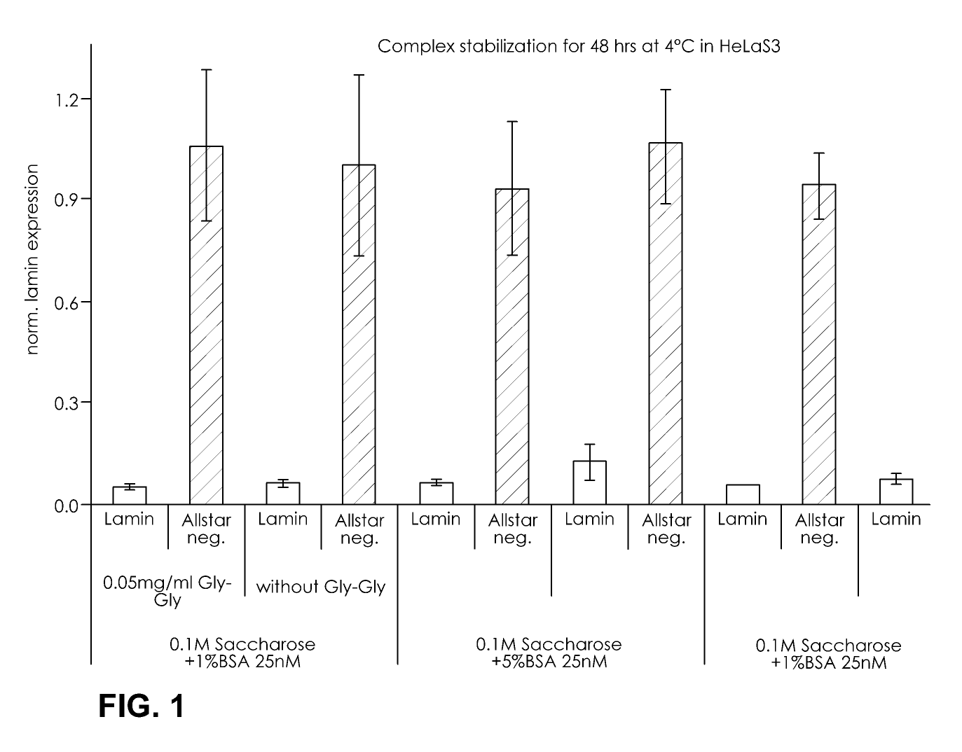

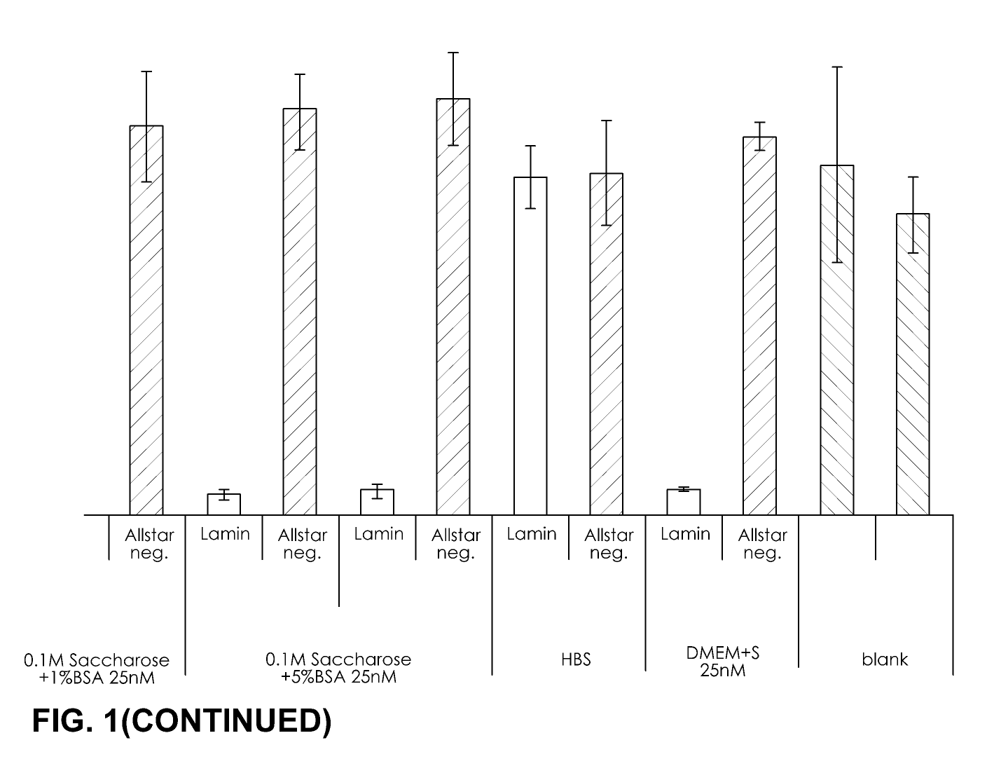

The expression of lamin normalized to GAPDH is shown in FIG. 1. The light bars show the expression of the lamin mRNA in cells which had been transfected with lamin A/C (SEQ ID NO: 9) siRNA, and the dark bars show the expression of the lamin mRNA in cells which had been transfected with NSC (SEQ ID NO: 15) siRNA, abbreviated as Allstar neg. in the figure.

Under the bars, the respective compositions of the stabilizer are stated, no stabilizing components were added to HBS, and the null control is designated by “blank”.

FIG. 1 shows that after storage of the siRNA with the transfection reagent HiPerFect for 2 days at 4° C. no HeLa S3 cells can any longer be successfully transfected. The normalized lamin expression lies in the region of the expression of the Non Silencing Control (Allstar neg.). On the other hand, if stabilizing compositions were added to the complex of transfection reagent and siRNA, then after the storage a successful transfection of the lamin siRNA was possible. Thus it was shown that at all concentrations used the combination of saccharose and BSA had the same stabilizing effect on the complex of transfection reagent and siRNA. The addition of Gly-Gly had neither a positive nor a negative effect on the stabilization. DMEM with serum, about 90% of the protein whereof consists of BSA, was also found suitable for stabilizing the complex in this experiment.

This experiment showed that it is possible to store a complex of transfection reagent and siRNA for 2 days at 4° C. without the transfection efficiency being reduced, if a mixture of saccharose and BSA is added to this complex. If these two components are lacking, transfection after storage is no longer possible.

### Example 2

### Stabilization of a Complex of Transfection Reagent and siRNA with Various Compositions with Storage of the Complex for 5 Days at 4° C. and at Room Temperature

The same procedure as described in example 1 was used, except only that this time the complex of siRNA and transfection reagent was stored for 5 days at 4° C. or at room temperature.

The following compositions were used as stabilizers of the complex of siRNA and transfection reagent:

0.1M saccharose

1% (w/v) BSA

5% (w/v) BSA

Buffer 1B: 0.1M saccharose, 1% (w/v) BSA

Buffer 2B: 0.1M saccharose, 5% (w/v) BSA

DMEM without serum

DMEM with serum

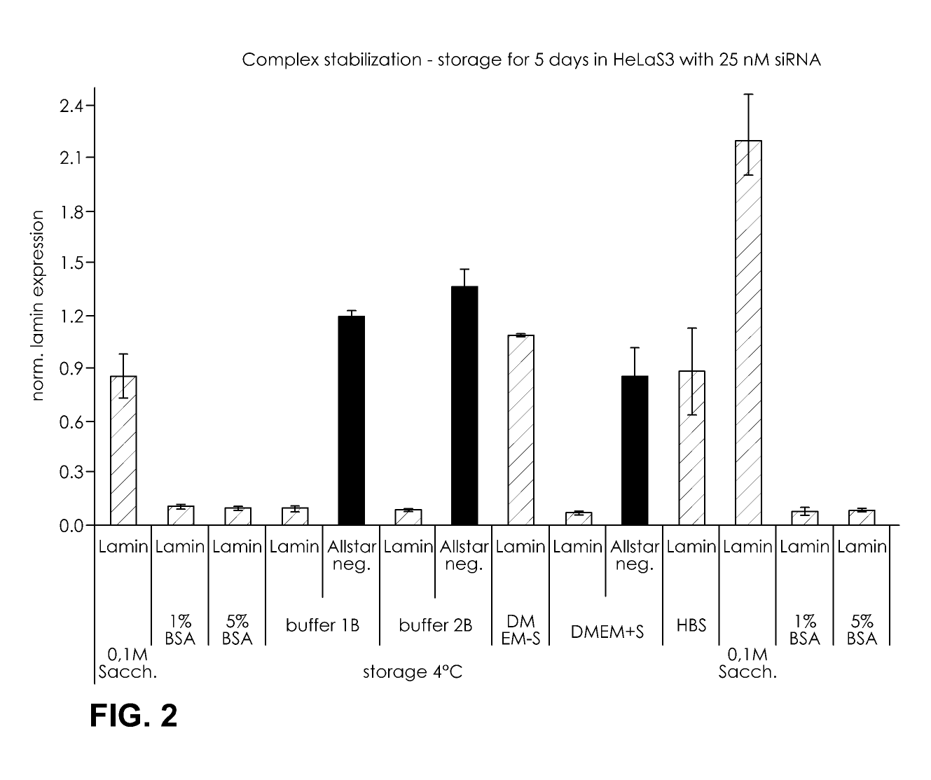

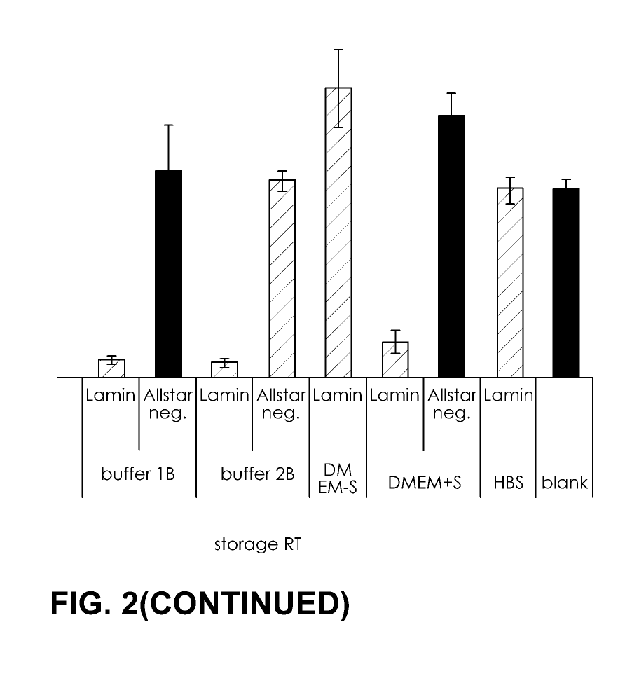

The result of this experiment is reproduced in FIG. 2. FIG. 2 shows that it is possible to stabilize the complex of siRNA and transfection reagent for 5 days such that it is possible to transfect this complex after storage. Here it is unimportant whether the complexes had been stored at 4° C. or at room temperature, the transfection efficiency is comparable.

As well as a mixture of saccharose and BSA, under these experimental conditions BSA alone is also capable of stabilizing the complex of siRNA and transfection reagent. DMEM with serum is also suitable for this, but not saccharose alone or DMEM without serum.

### Example 3

### Stabilization of a Complex of Transfection Reagent and siRNA with Various Compositions with Storage of the Complex for 2 Days at 4° C. with Different Concentrations of siRNA Used

The same procedure as described in example 1 was used, except only that this time in addition to 25 nM siRNA, smaller quantities of siRNA (10 nM, 5 nM, 1 nM, 0.1 nM) were also used. This time the storage took place for 2 days at 4° C.

The following compositions were used as stabilizers of the complex of siRNA and transfection reagent:

1% (w/v) BSA

5% (w/v) BSA

DMEM with serum

As the control, a transfection was performed with freshly prepared transfection complexes in DMEM without serum.

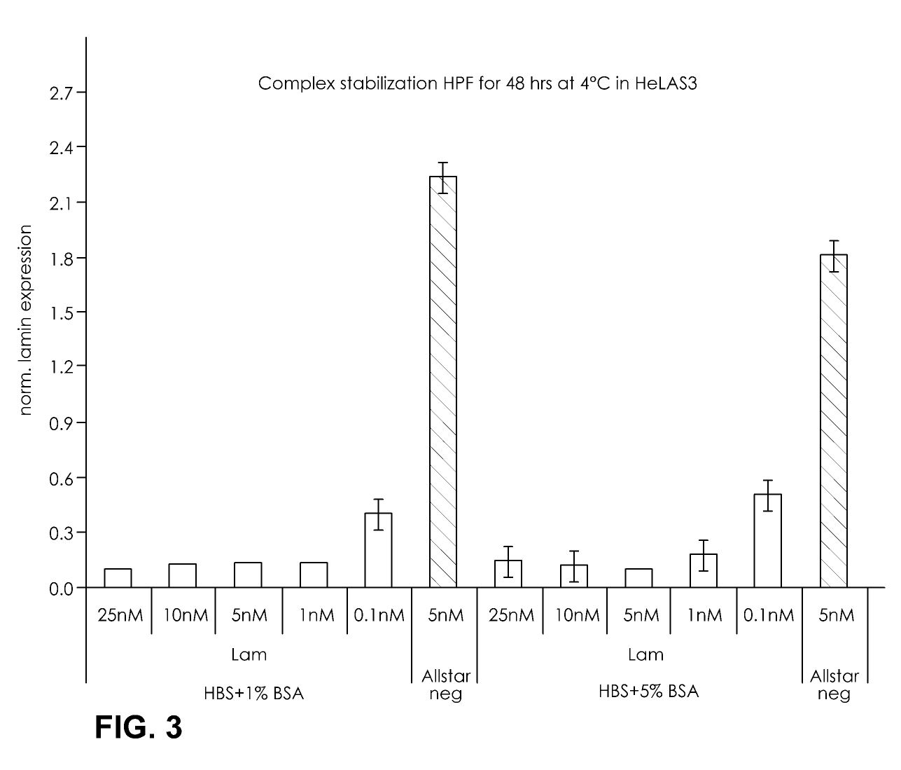

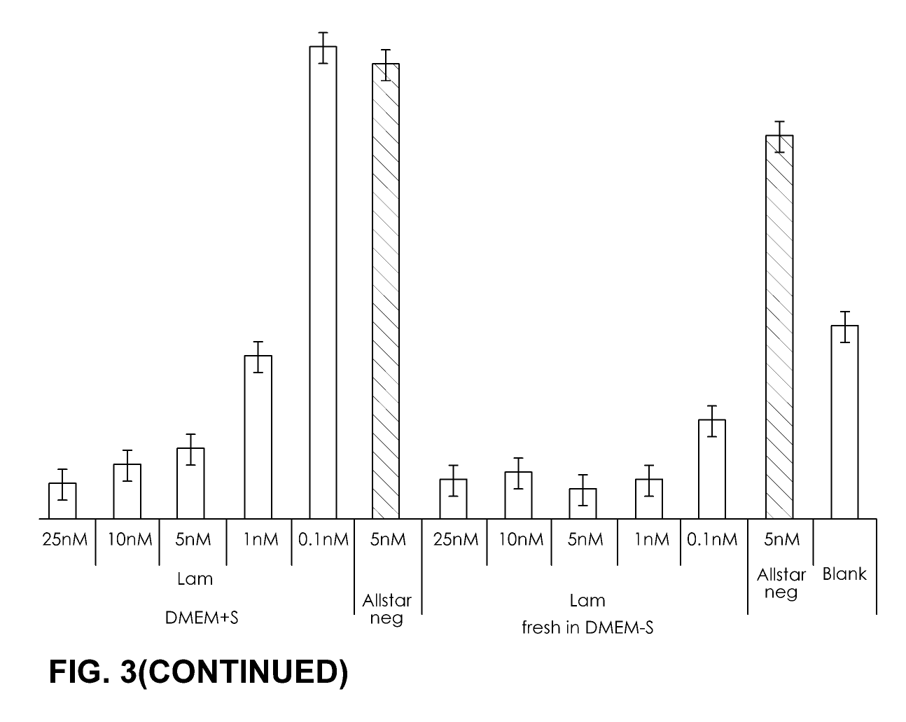

The result of this experiment is shown in FIG. 3. Here it was found that the transfection efficiency after storage in BSA is about as good to slightly better than with freshly prepared transfection complexes. On the other hand, on storage in DMEM with serum, a markedly worse transfection efficiency was found, particularly when working with lower siRNA concentrations (less than 5 nM).

### Example 4

### Stabilization of a Complex of Transfection Reagent and siRNA with Various Compositions with Storage of the Complex for 5 Days at 4° C. with Different Concentrations of siRNA Used

The same procedure as described in example 3 was used, except only that this time the storage was effected for 5 instead of 2 days at 4° C., and a control with freshly prepared transfection complexes was dispensed with.

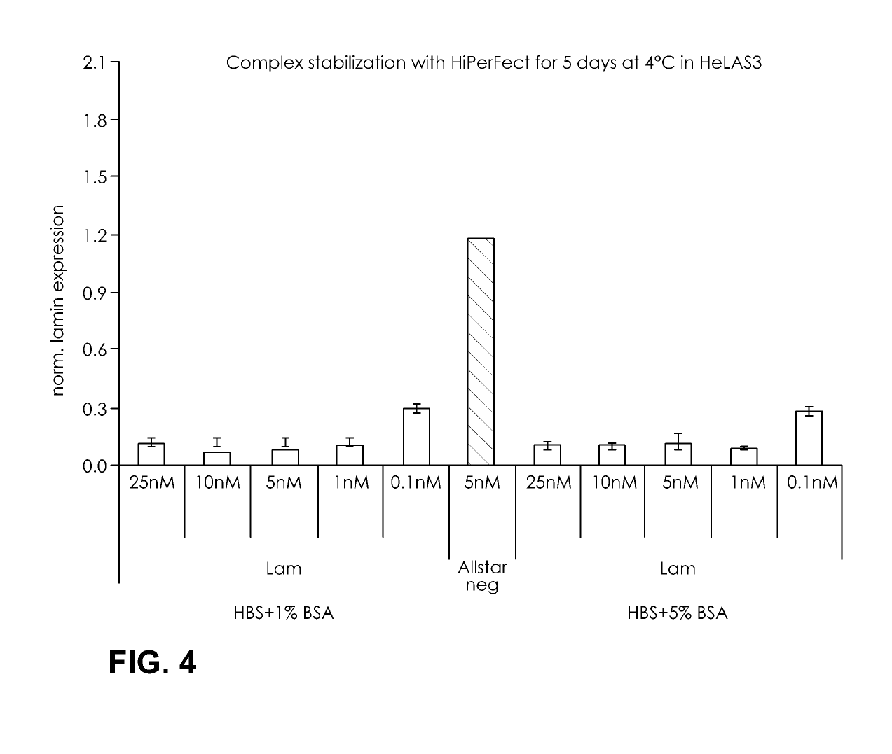

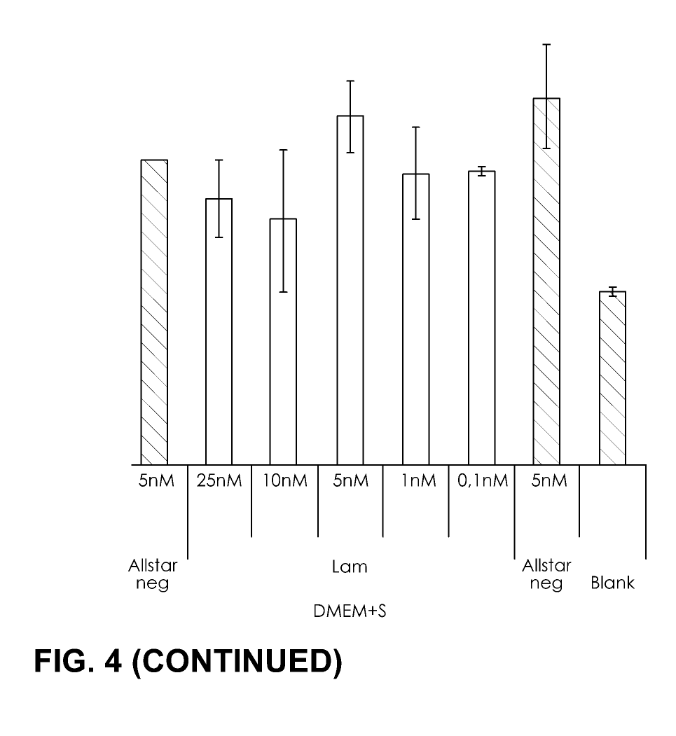

The result of this experiment is shown in FIG. 4. FIG. 4 shows that the transfection efficiency with 1% and 5% BSA as stabilizer remained unchanged and high after 5 days, so that no adverse effects of the storage on the transfection efficiency are seen. In contrast, successful transfection can no longer be effected if the complex of siRNA and transfection reagent has been stored for 5 days at 4° C. in DMEM with serum.

### Example 5

### Drying of a Complex of Transfection Reagent and siRNA without Stabilizing Composition

The same procedure as described in example 1 was used, except only that this time a mixture of Hs_ubb (SEQ ID NO: 14) and Hs_plk1 (SEQ ID NO: 13) siRNAs was used as siRNA. These two siRNAs serve for the silencing of two different genes in a eukaryotic cell for the induction of cell death. They are Cell Death Control siRNA. The quantities 6.6 nM, 16.6 nM and 33.3 nM siRNA were used. As transfection reagent, 0.75 and 1.5 μl of HiPerFect (QIAGEN) were used.

Other than as described in example 1, in this example no composition stabilizing the complex of transfection reagent and siRNA was used, but only transfection reagent and siRNA in buffer HBS. The complex was dried overnight and then taken up again in 100 μl of water. For comparison, the NSC (SEQ ID NO: 15) siRNA (Allstar neg.), which exerts no silencing effect in cells, was again used as the null control.

The transfection was performed as described in example 1. As the control, transfection was also performed with freshly prepared complexes of transfection reagent and siRNA which had not been dried beforehand.

3 days after the transfection, the number of surviving cells was determined in all preparations as per the protocol of the CellTiter Glo Luminescent Cell Viability Assay (Promega). The more living cells are present in the preparation, the higher are the measurable light units (luminescence).

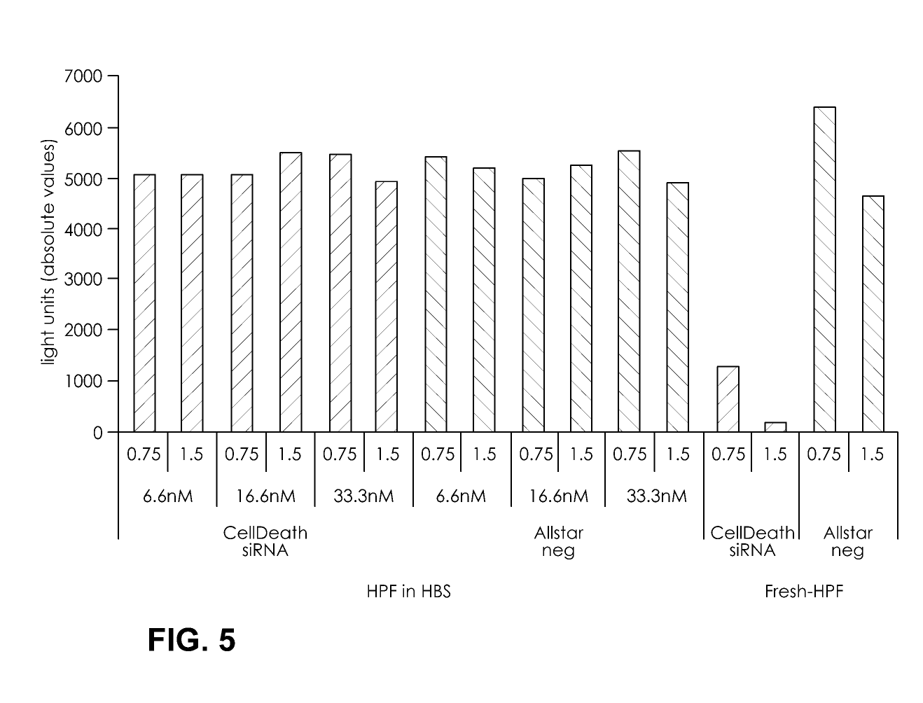

FIG. 5 shows that only in the freshly prepared transfection preparations which had not been dried beforehand was cell death induced by the Cell Death Control siRNA, but not in the preparations wherein the complex of siRNA and transfection reagent had been dried beforehand. This shows that the result of drying without stabilizing composition is that successful transfection can no longer be performed.

### Example 6

### Stabilization of a Complex of Transfection Reagent and siRNA with Stabilizing Composition Through Drying and Storage of the Complex for 2 Months at 4° C., Room Temperature and 37° C.

The same procedure as described in example 1 was used, except only that as the stabilizing composition only HBS buffer with 1% (w/v) BSA and 0.1M saccharose in a volume of 12.5 μl was used. In this experiment, two different siRNAs were used which are directed against the transcript of WASL, WASL_{—}6 (SEQ ID NO: 11) and WASL_{—}9 (SEQ ID NO: 12). Concerning WASL_{—}6 (SEQ ID NO: 11) it is known that it is less functional than WASL_{—}9 (SEQ ID NO: 12). In addition, the NSC (SEQ ID NO: 15) siRNA was used as the negative control. As cell lines this time, in addition to HeLa S3, the cell lines MCF-7 and HepG2 were used.

The complexes of transfection reagent and siRNA were mixed together with the stabilizing composition and dried overnight in vacuo. Next, the dried complex was stored for 2 months at 4° C., room temperature or 37° C. and then taken up again in 12.5 μl of water, before the different cell lines were directly transfected.

As primers for the quantitative RT-PCR, WASLF (SEQ ID NO: 5) and WASLR (SEQ ID NO: 6) were used.

As the control, freshly prepared transfection complexes which had been neither dried nor stored were transfected.

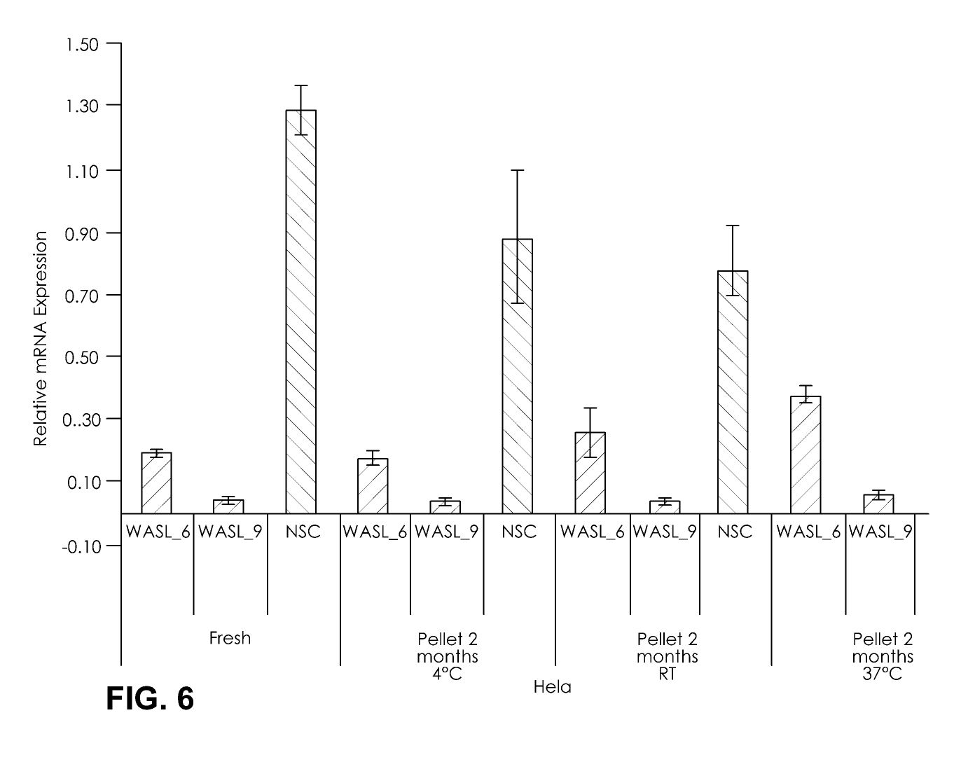

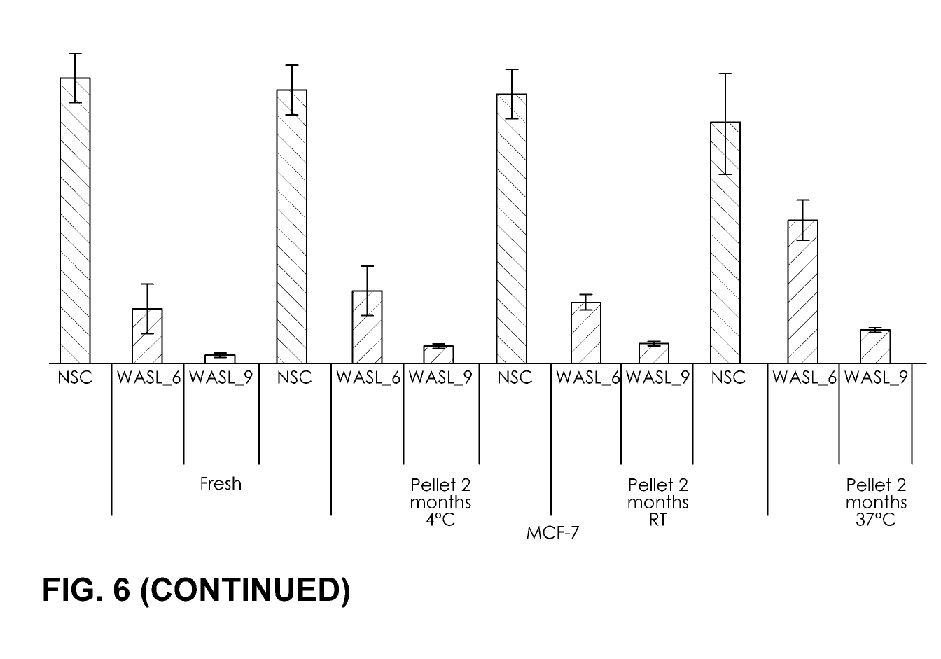

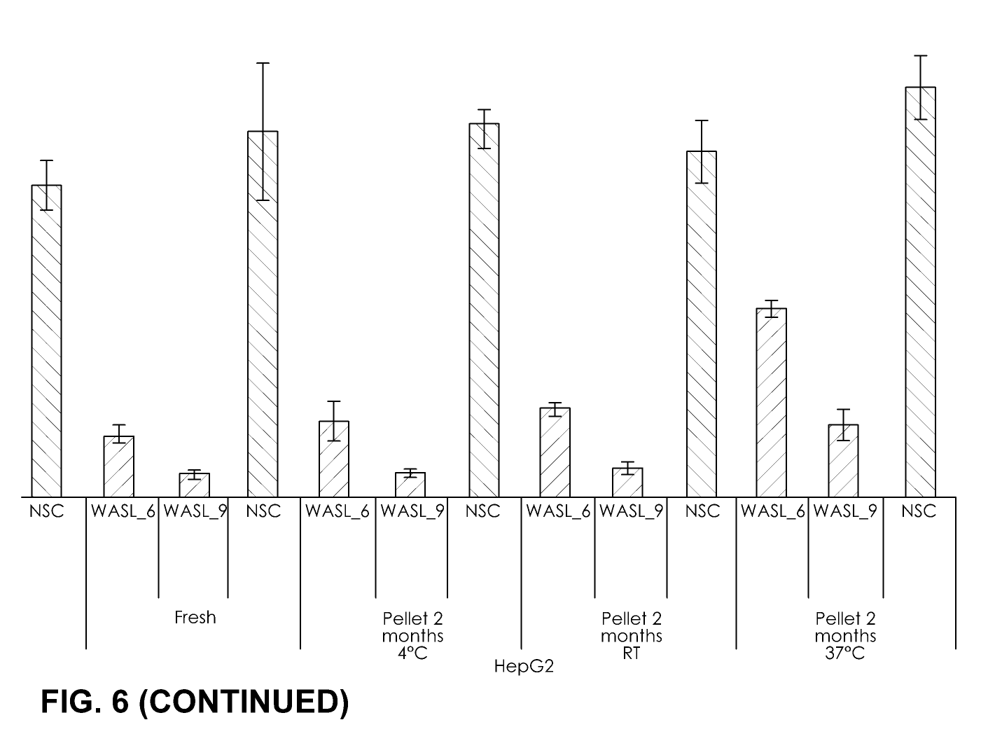

The result of this experiment is shown in FIG. 6. Here it was found that in all three cell lines comparable results had been obtained. The complexes stored for two months with the stabilizing composition were in all cases suitable for performing a successful transfection after the storage. At the same time it was found that the efficiency of the transfection with storage at 4° C. with all three cell lines gave identical results to transfecting freshly prepared complexes. With storage at room temperature, the efficiency was only minimally worse than with storage at 4° C., and even storage at 37° C. for 2 months still gave a very efficient transfection, which was however somewhat less efficient compared with the storage at 4° C. and at room temperature.

If a complex of transfection reagent and siRNA is dried in presence of a mixture of BSA and saccharose, then it is stabilized thereby such that the transfection efficiency is not reduced after 2 months' storage. In contrast to this, example 5 showed that the transfection efficiency approaches zero when the complex of transfection reagent and siRNA is dried without the presence of mixture of protein and saccharide.

### Example 7

### Stabilization of a Complex of Transfection Reagent and siRNA with Stabilizing Composition after Several Freeze-Thaw Cycles

The same procedure as described in example 1 was used, except only that this time in addition to HeLa S3 cells, MCF-7 cells were also used for the transfection and that as the stabilizing composition HBS buffer with 1% (w/v) BSA and 0.1M saccharose was used.

The complexes of transfection reagent, siRNA and stabilizing composition were frozen for 1 hour at −20° C. and then thawed for 1 hour at room temperature. This cycle was repeated three to five times. As the control, freshly prepared transfection complexes which had not been frozen were transfected.

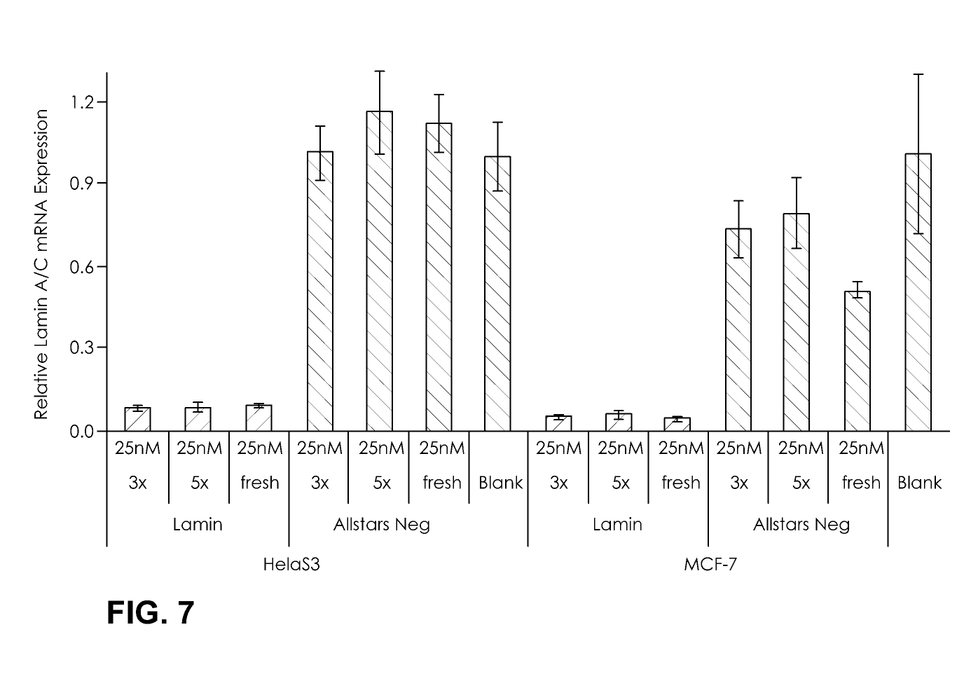

The result of this experiment is shown in FIG. 7. Here it was found that multiple freezing and thawing of the transfection complexes with the stabilizing composition enables just as efficient a transfection as a transfection with a freshly prepared transfection complex.

This experiment shows that the complex of transfection reagent and nucleic acid can be stored in the frozen state without problems, when a composition of protein and saccharide stabilizes the complex.

### Example 8

### Stabilization of a Complex of Transfection Reagent and siRNA with Stabilizing Composition and Different Transfection Reagents

The same procedure as described in example 1 was used, except only that this time in addition to the transfection reagent HiPerFect, the transfection reagent HT-Fect (QIAGEN) was used. As siRNA this time, CDC2 was used in addition to NSC (SEQ ID NO: 15), and as the primers in the quantitative RT-PCR the primers CDC2F (SEQ ID NO: 3) and CDC2R (SEQ ID NO: 4) were used.

As stabilizing compositions, the following were used:

Buffer A: 0.1M saccharose, 1% (w/v) BSA in HBS

Buffer B: 0.1M saccharose, 0.4% (w/v) BSA in HBS

Buffer C: 0.25 M trehalose, 1% (w/v) BSA in HBS

Buffer D: 0.1M trehalose, 0.4% (w/v) BSA in HBS

The complex of transfection reagent and siRNA in the presence of the stabilizing composition was stored either dried or at −20° C. undried. Before the transfection, the complexes were respectively again taken up in water or thawed. As the control, freshly prepared complexes of transfection reagent and siRNA were transfected.

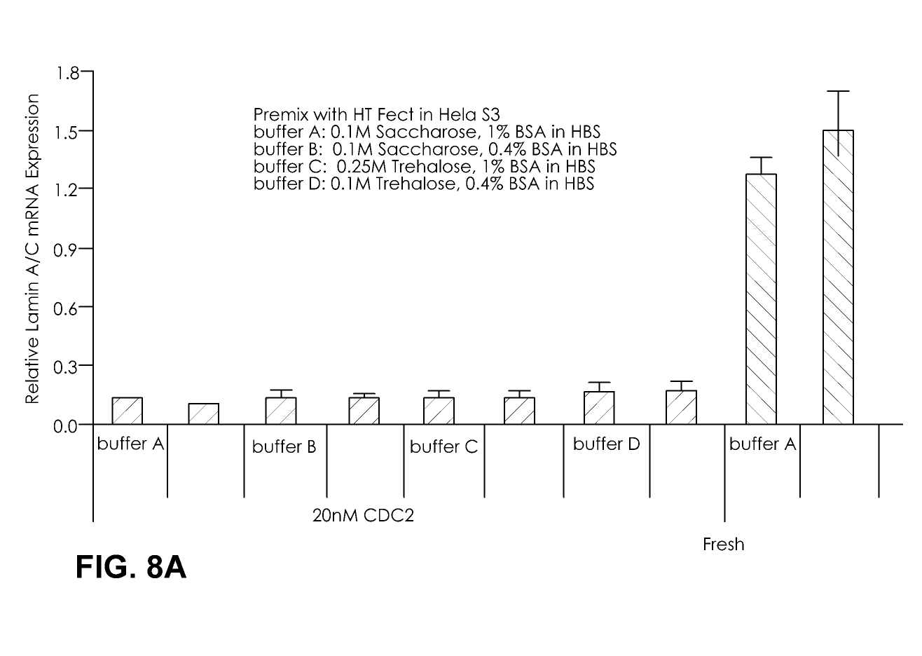

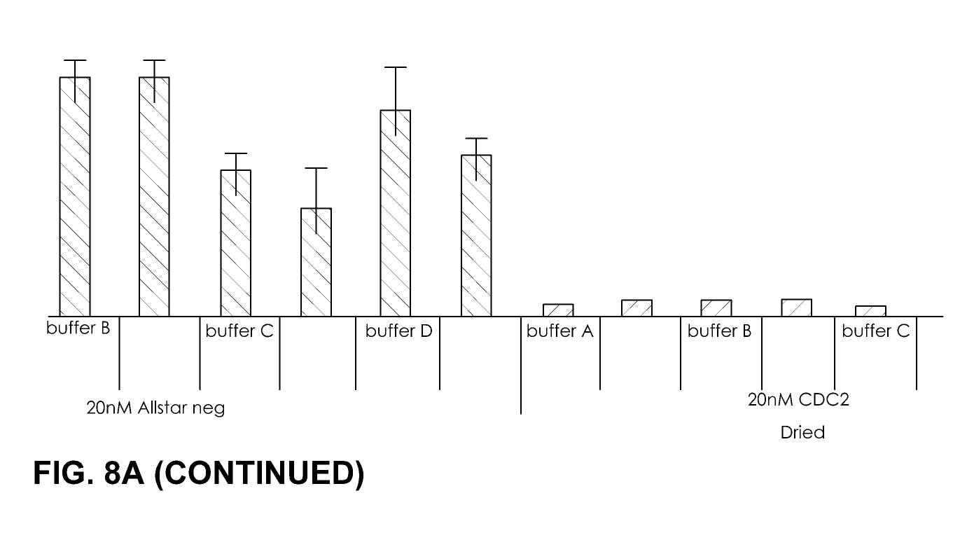

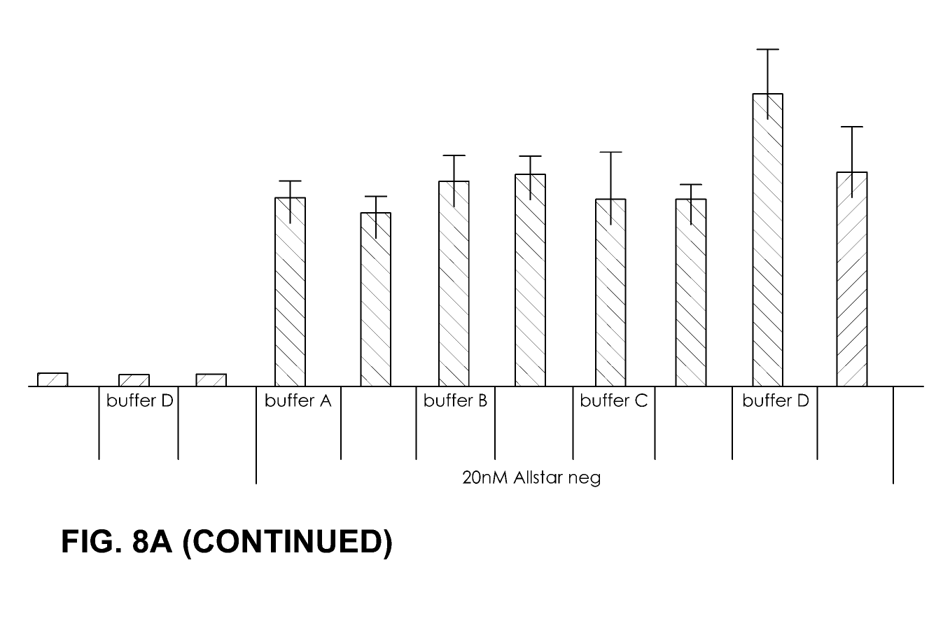

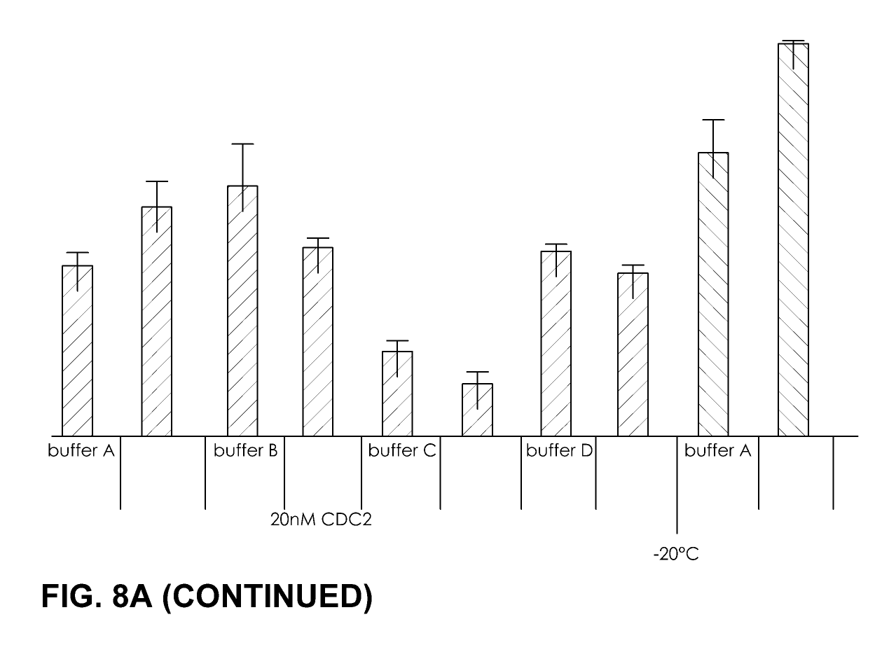

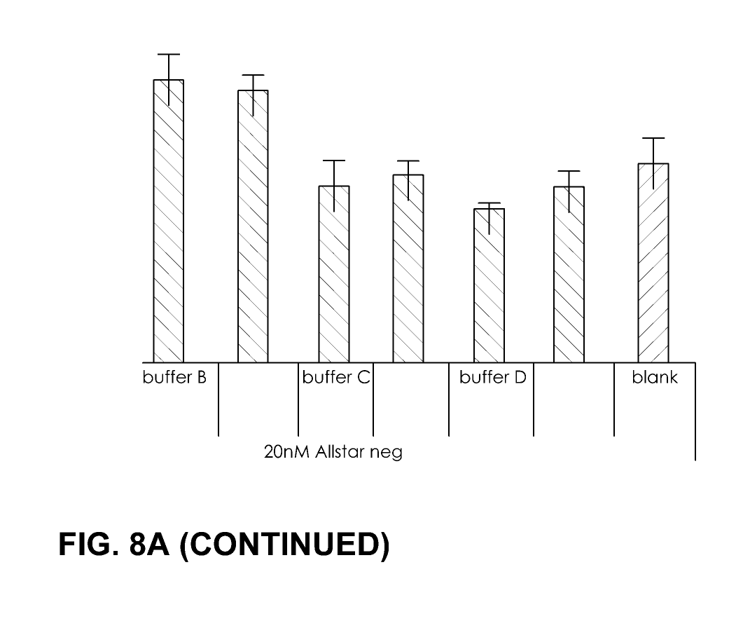

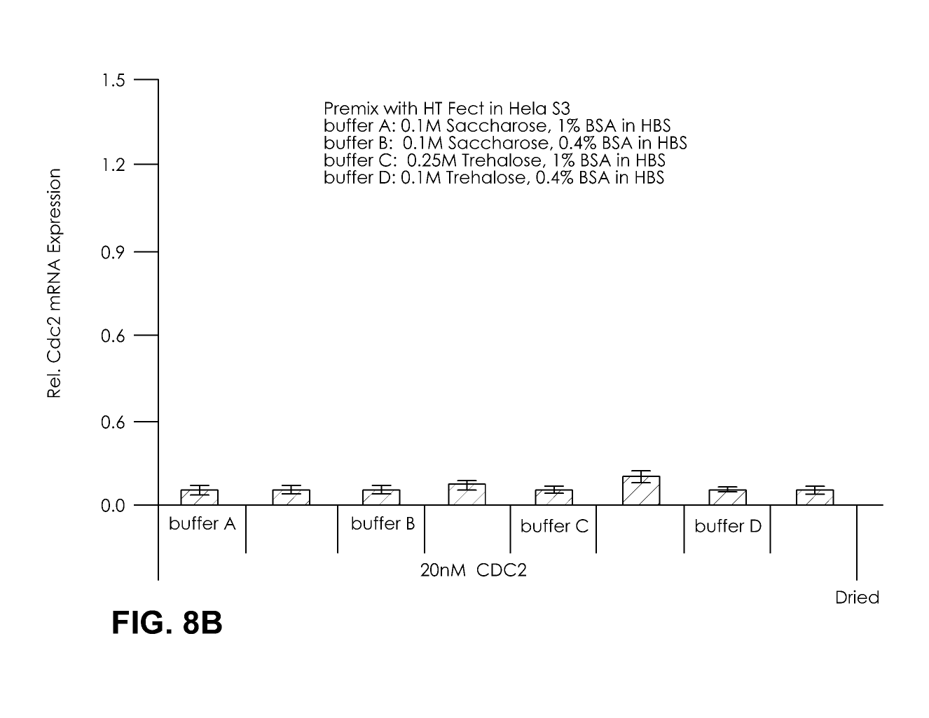

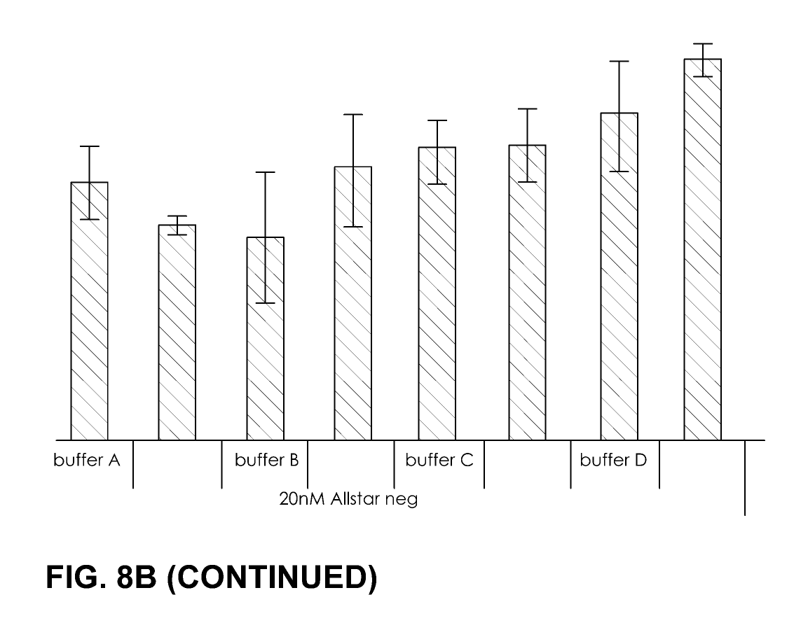

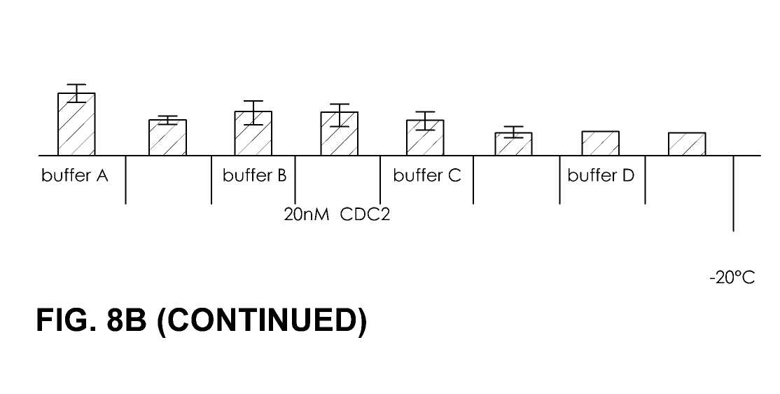

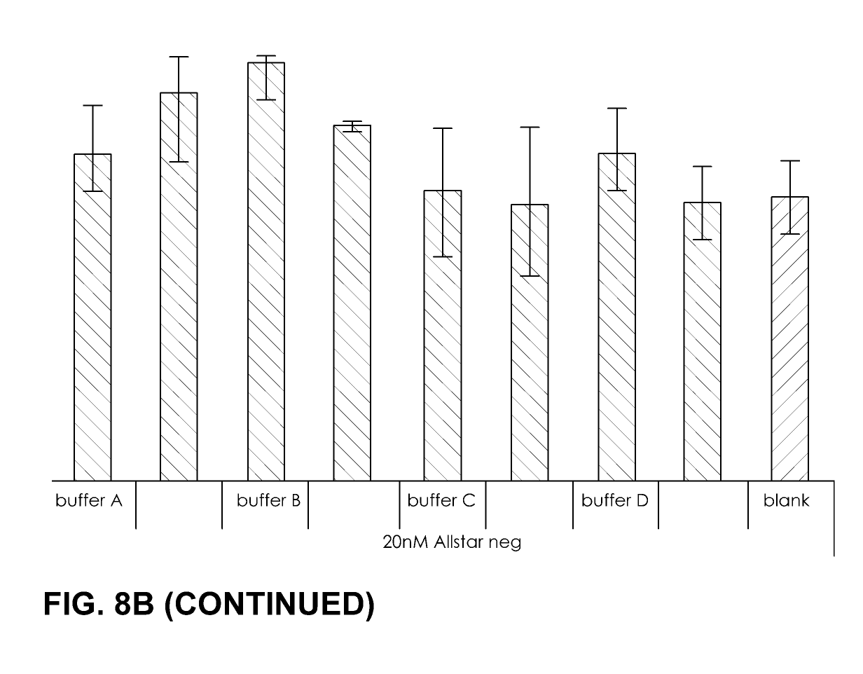

The result of this experiment is shown in FIG. 8A for HT Fect and in FIG. 8B for HiPerFect.

The experiment showed that the stabilizing property of the composition is not limited only to the transfection reagent HiPerFect, but that other transfection reagents can also be stabilized in their complex with the nucleic acid by means of the composition. Further, the experiment showed that saccharides other than saccharose can also be used in the composition.

FIG. 8A shows that after the transfection with HT Fect the CDC2 mRNA is down-regulated to a comparable extent when transfection was performed fresh or when the complex of transfection reagent and nucleic acid was dried beforehand. However, if the complex of transfection reagent and nucleic acid was frozen at −20° C., then only the composition buffer C is still suitable for subsequently performing a successful transfection with HT Fect, and with the other three compositions studied no successful transfection could be observed after the freezing at −20° C., if the transfection was performed with HT Fect.

If on the other hand the transfection was performed with HiPerfect (FIG. 8B), then it was found that all four compositions studied, both dried and also stored at −20° C., gave a comparable transfection efficiency to freshly prepared transfection complexes. Also, the results for saccharose and for trehalose were comparable.

This experiment showed that other saccharides than saccharose mixed with a protein are also suitable for stabilizing a complex of transfection reagent and nucleic acid.

Further, this experiment showed that the stabilizing action of the composition is not limited to a single transfection reagent, and the experiment has also shown that there are transfection reagents for which the stabilizing composition can be more suitable than for others.

### Example 9

### Stabilization of a Complex of Transfection Reagent and Plasmid DNA

2, 3 or 4 μl of HiPerfect were mixed together with 400 ng of plasmid DNA which had an open reading frame for Green Fluorescent Protein (pGFP) in 50 μl of HBS buffer. In addition, 0.1M saccharose and 1% (w/v) BSA were added (Premix) to the preparations as stabilizing composition.

After the complex formation for 10 mins at room temperature, the complexes were dried overnight in vacuo and stored in the dried state at 4° C. for 4 hours. Before the transfection, the complexes were taken up in water and transfected as stated in example 1. In contrast to example 1, this time plasmid DNA and no siRNA were transfected and, in addition to HeLa S3 cells, MCF-7 cells were also transfected with the plasmid DNA. For comparison, the plasmid DNA was also transfected in the fresh transfection complex with HiPerFect (Standard).

After an incubation of the transfection preparation for 2 days at 37° C. and 5% CO₂, the number of transfected cells was measured by fluorescence-activated cell sorting (FACS) in an FACSCalibur (Becton Dickinson). All cells which have been successfully transfected with the pGFP plasmid express the GFP, which emits a fluorescence measurable in the FACS.

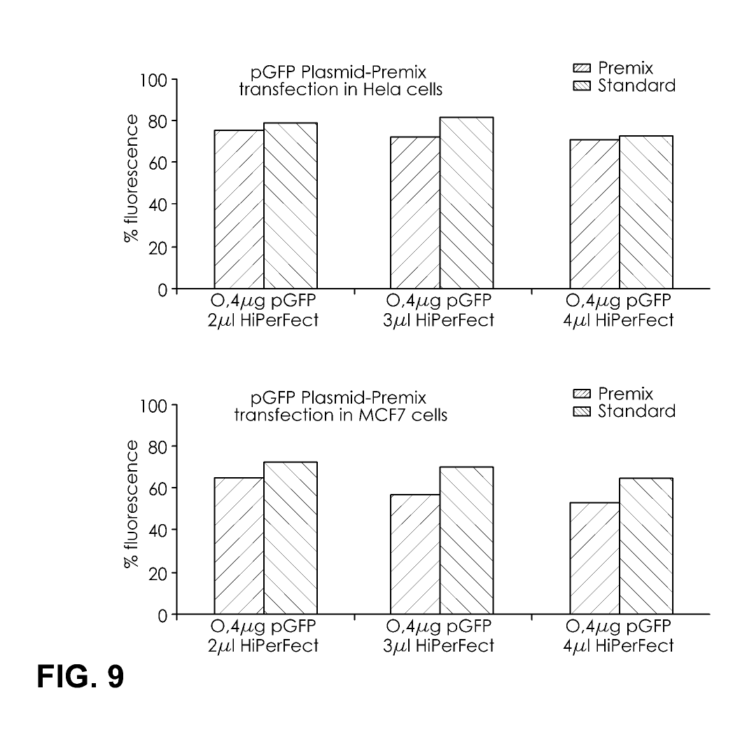

In FIG. 9, the result of the FACS analysis is shown. At all three tested concentrations of transfection reagent, the transfection efficiency in HeLa S3 cells is very high and almost identical when the transfection took place after drying of the complex with stabilizing composition or fresh without stabilizing composition.

With MCF-7 cells, the transfection efficiency is also very high, and the efficiency of the transfection after drying is only slightly lower than with the fresh preparation.

This experiment shows that the stabilizing composition is suitable not only for stabilizing RNA with a transfection reagent, but also to the same extent for stabilizing DNA with a transfection reagent.

What is claimed is:

1. A method for transfecting a eukaryotic cell comprising:
contacting a complex of a nucleic acid and a transfection reagent that is in a dried state with a liquid to bring the complex into a liquid state; and
contacting the eukaryotic cell with the complex in the liquid state to effectuate transfection of the eukaryotic cell with the nucleic acid;
wherein the complex, prior to being dried, was formed by contacting a composition comprising at least one saccharide and at least one protein and optionally a salt and/or a pH-stabilizing component with the nucleic acid and the transfection reagent.

2. The method according to claim 1, wherein the complex is stored in the dried state for at least two days before being contacted with the liquid.

3. The method according to claim 1, wherein drying of the composition, nucleic acid and transfection reagent is conducted in vacuo.

4. The method according to claim 3, wherein drying is effected by a vacuum bell jar, by vacuum centrifugation or by a freeze-drying unit.

5. The method according to claim 1, wherein drying of the composition, nucleic acid and transfection reagent is conducted by evaporation.

6. The method according to claim 5, wherein evaporation is conducted at a temperature above 25° C. but below a temperature where the nucleic acid or the transfection reagent would be thermally destroyed or irreversibly denatured.

7. The method according to claim 1, wherein the liquid is water.

8. The method according to claim 1, wherein the liquid is a buffer which contains salts and/or a pH-stabilizing component.

9. The method according to claim 2, wherein the complex is stored at a temperature within the range of from about 20° C. to about 25° C. for at least some time after drying and before contacting the liquid.

10. The method according to claim 1, wherein the complex is shipped from a first location to a second location while in the dried state.

11. The method according to claim 1, wherein the eukaryotic cell is a cell culture cell.

12. The method according to claim 1, wherein the nucleic acid is a ribonucleic acid.

13. The method according to claim 12, wherein the nucleic acid is a double-stranded ribonucleic acid which has a length of about 18 to 30 nucleotides.

14. The method according to claim 1, wherein the transfection reagent is a lipid-based transfection reagent.

15. The method according to claim 1, wherein the saccharide is a monosaccharide, a disaccharide, a trisaccharide, a polysaccharide or a mixture of two or more thereof.

16. The method according to claim 1, wherein the saccharide is a disaccharide selected from the group consisting of saccharose, trehalose and mixtures thereof.

17. The method according to claim 1, wherein the saccharide is present in the composition at a concentration of 0.01M to 2M.

18. The method according to claim 1, wherein the protein is selected from the group consisting of bovine serum albumin, fetal calf serum and mixtures thereof.

19. The method according to claim 1, wherein the protein is accounts for about 0.1% to 5% of the composition by weight.

20. A method for transfecting a eukaryotic cell comprising:
contacting a complex of a nucleic acid and a transfection reagent that is in a dried state with a liquid to bring the complex into a liquid state; and
contacting the eukaryotic cell with the complex in the liquid state to effectuate transfection of the eukaryotic cell with the nucleic acid;
wherein the complex, prior to being dried, was formed by contacting a composition comprising at least one saccharide and at least one protein and optionally a salt and/or a pH-stabilizing component with the nucleic acid and the transfection reagent,
wherein the eukaryotic cell is a cell culture cell,
wherein the nucleic acid is a double-stranded ribonucleic acid which has a length of about 18 to 30 nucleotides,
wherein the transfection reagent is a lipid-based transfection reagent,
wherein the saccharide is a disaccharide,
wherein the disaccharide is present in the composition at a concentration of 0.01M to 2M,
wherein the protein is bovine serum albumin,
wherein the bovine serum albumin accounts for about 0.1% to 5% of the composition by weight,
wherein the complex is stored in the dried state for at least two months before being contacted with the liquid,
wherein the complex is stored at a temperature within the range of from about 20° C. to about 25° C. for at least some time after drying and before contacting the liquid, and
wherein the complex is shipped from a first location to a second location while in the dried state.


SEQUENCE LISTING (15 sequences)

SEQ ID NO: 1 - DNA, 22 bp, Artificial
  Oligonucleotide primer sequence
gcatctcatcctgaagttgctt

SEQ ID NO: 2 - DNA, 20 bp, Artificial
  Oligonucleotide primer sequence
caagaaggagggtgacctga

SEQ ID NO: 3 - DNA, 21 bp, Artificial
  Oligonucleotide primer sequence
caggattttcagagctttggg

SEQ ID NO: 4 - DNA, 27 bp, Artificial
  Oligonucleotide primer sequence
gctggatcatagattaacattttcgag

SEQ ID NO: 5 - DNA, 21 bp, Artificial
  Oligonucleotide primer sequence
cgacaaaggaaatctgagaaa

SEQ ID NO: 6 - DNA, 21 bp, Artificial
  Oligonucleotide primer sequence
gggagatgttgttgacttgtg

SEQ ID NO: 7 - DNA, 20 bp, Artificial
  Oligonucleotide primer sequence
gcctcaagatcatcagcaat

SEQ ID NO: 8 - DNA, 20 bp, Artificial
  Oligonucleotide primer sequence
gagtccttccacgataccaa

SEQ ID NO: 9 - DNA, 21 bp, Artificial
  siRNA
gcgccagaauggagaugautt

SEQ ID NO: 10 - RNA, 21 nt, Artificial
  siRNA
uugaguaacgagcugacccca

SEQ ID NO: 11 - DNA, 20 bp, Artificial
  siRNA
uuuuaaacucccuuguugat

SEQ ID NO: 12 - DNA, 21 bp, Artificial
  siRNA
uuuguuuccugcaguaguugg

SEQ ID NO: 13 - RNA, 21 nt, Artificial
  siRNA
uauucauucuucuugauccgg

SEQ ID NO: 14 - DNA, 21 bp, Artificial
  siRNA
ggccaagauccaagauaaatt

SEQ ID NO: 15 - RNA, 21 nt, Artificial
  siRNA
uuuguaaucgucgauacccug